(19) United States
(12) Patent Application Publication
(10) Pub. No.: US 2009/0202442 A1
(43) Pub. Date: Aug. 13, 2009

(54) Enerceutical activation of the alternative cellular energy (ACE) pathway in therapy of diseases

(76) Inventor: William John Martin, South Pasadena, CA (US)

Correspondence Address: William John Martin, 1634 Spruce Street, South Pasadena, CA, 91030, US

(21) Appl. No.: 12/069,597

(22) Filed: Feb. 11, 2008

(51) Int. Cl. A61K 49/00; A61K 31/164; A61P 43/00

(52) U.S. Cl. 424/9.6; 514/626

(57) ABSTRACT

Alternative cellular energy pigments (ACE-pigments) provide a source of cellular energy other than that provided through the oxidative metabolism of foods, or in the case of plants and certain bacteria, through the process of photosynthesis. In some patients, ACE pigments exist in a form that can be further energized or activated using ultraviolet (UV) light, especially if the reaction is initially triggered by the presence of suitable dyes, such as neutral red. A method is described to further enhance the activation of the ACE pathway in humans and animals deprived of ACE. The method comprises using natural or man-made sources of ACE products (enerceuticals), with or without the inclusion of a suitable dye, such as neutral red; and applying the material(s) to the skin, either directly or separated by an impermeable barrier; and illuminating the enerceutical with a UV light source. The process of activating the ACE pathway is evidenced by UV inducible fluorescence seen within areas of the patients' skin and/or mucus membranes. This fluorescence fades as the ACE pathway becomes fully activated. Activating the ACE pathway can have therapeutic benefits in various infectious and non-infectious diseases. Cited examples include autism, Morgellons disease, herpes virus infections and cellulite.

### CROSS REFERENCE TO RELATED APPLICATIONS

### Co-Pending Patent Application

### Methods for Detection of Ultraviolet Light Reactive Alternative Cellular Energy Pigments (ACE-pigments) William John Martin Submitted Dec. 24, 2007

### Method of Activating the Alternative Cellular Energy (ACE) Pathway in the Therapy of Herpes Virus Infections William John Martin and Sheila Calderon Submitted Dec. 26, 2007

Method of Assessing and of Activating the Alternative Cellular Energy (ACE) Pathway in the Therapy of Diseases. William John Martin Submitted Jan. 17, 2008

### Previously Submitted but Now Abandoned Patent Application

Ser. No. 10/044,683. Therapy of stealth virus associated cancers and other conditions using light. William John Martin. (Abandoned)
Ser. No. 10/047,313. Therapy of stealth virus associated cancers and other conditions using medium chain triglycerides. William John Martin. (Abandoned)
Ser. No. 10/050,232. Diagnosing and monitoring the therapy of stealth virus infections based on the detection of auto-fluorescent material in hair. William John Martin. (Abandoned)
Ser. No. 10/058,480. Therapy of stealth virus associated cancers and other conditions using magnetic energy. William John Martin. (Abandoned)
Ser. No. 10/164,258 Energy supportive therapy of stealth virus associated diseases. William John Martin. (Abandoned)
Ser. No. 10/174,466 Sound therapy of stealth virus associated diseases. William John Martin. (Abandoned)
Ser. No. 10/192,936 ACE-Pigments and humic acids as energy sources. William John Martin. (Abandoned)
Submitted: Ser. No. 10/______, Methods for Collection of Alternative Cellular Energy Pigments (ACE-pigments). William John Martin. (Abandoned)
Submitted: Ser. No. 10/______, Methods for Elimination of Toxic Alternative Cellular Energy Pigments (ACE-pigments) and for Their Replacement Using Activated Humates, Including Humic and Fulvic Acids. William John Martin. (Abandoned)

### United States Patents (Awarded)

U.S. Pat. No. 5,985,546 Stealth virus detection in the chronic fatigue syndrome. William John Martin
U.S. Pat. No. 5,891,468 Stealth virus detection in the chronic fatigue syndrome. William John Martin
U.S. Pat. No. 5,753,488 Isolated stealth viruses and related vaccines. William John Martin
U.S. Pat. No. 5,703,221 Stealth virus nucleic acids and related methods. William John Martin

### PCT (Patent Cooperation Treaty)

WO 92/20797 Stealth virus detection in the chronic fatigue syndrome. William John Martin
WO 99/34019 Stealth virus nucleic acids and related methods. William John Martin
WO 99/60101 Stealth viruses and related vaccines. William John Martin

### REFERENCES TO PUBLISHED ARTICLES

### Alternative Cellular Energy Pigments (ACE-pigments):

1 Martin W J. Alternative cellular energy pigments mistaken for parasitic skin infestations. Exp. Mol. Path 78: 212-214, 2005.
2 Martin W J. Alternative cellular energy pigments from bacteria of stealth virus infected individuals. Exp. Mol. Path 78: 217-217, 2005.
3 Martin W J. Progressive Medicine. Exp Mol Path 78: 218-220, 2005.
4 Martin W J, Stoneburner J. Symptomatic relief of herpetic skin lesions utilizing an energy based approach to healing. Exp. Mol. Path 78: 131-4, 2005.
5 Martin W J. Etheric Biology. Exp Mol Path 78: 221-227, 2005.
6 Martin W J. Stealth Virus Culture Pigments: A Potential Source of Cellular Energy. Exp. Mol. Pathol. 74: 210-223, 2003.
7 Martin W J. Complex intracellular inclusions in the brain of a child with a stealth virus encephalopathy. Exp. Mol. Pathol. 74: 179-209, 2003.
8 Martin W J. Photons and phonons: Theoretical aspects of biophysics and potential therapeutic applications. Proceeding of Neural Therapy Workshop on Sound and Light Therapy, Seattle, Wash., Feb. 21-23, 2003.

### Stealth Adapted Viruses

1 Martin W J Chronic fatigue syndrome among physicians. A potential result of occupational exposure to stealth viruses. Explore 2001; 10: 7-10.
2 Martin W J. Stealth Viruses. Explore 2001; 10: 17-19.
3 Durie G M, Collins R. Martin W J. Positive stealth virus cultures in multiple myeloma. A possible explanation for neuropsychiatric co-morbidity. Presented at the Am. Soc. Hematology annual meeting October 2000.
4 Martin W J. Chemokine receptor-related genetic sequences in an African green monkey simian cytomegalovirus-derived stealth virus. Exp Mol Pathol. 2000; 69:10-6.
5 Martin W J., Anderson D. Stealth virus epidemic in the Mojave Valley: severe vaculating encephalopathy in a child presenting with a behavioral disorder. Exp Mol Pathol. 1999; 66:19-30.
6 Martin W J. Melanoma growth stimulatory activity (MGSA/GRO-alpha) chemokine genes incorporated into an African green monkey simian cytomegalovirus-derived stealth virus. Exp Mol Pathol. 1999; 66:15-8.
7 Martin W J. Bacteria-related sequences in a simian cytomegalovirus-derived stealth virus culture. Exp Mol Pathol. 1999; 66:8-14.
8 Martin W J. Stealth adaptation of an African green monkey simian cytomegalovirus. Exp Mol Pathol. 1999; 66:3-7.
9 Martin W J. Cellular sequences in stealth viruses. Pathobiology 1998; 66:53-8.
10 Martin W J. Detection of RNA sequences in cultures of a stealth virus isolated from the cerebrospinal fluid of a health care worker with chronic fatigue syndrome. Case report. Pathobiology. 1997; 65:57-60.
11 Martin W J., Anderson D. Stealth virus epidemic in the Mojave Valley. I. Initial report of virus isolation. Pathobiology. 1997; 65:51-6.
12 Martin W J. Simian cytomegalovirus-related stealth virus isolated from the cerebrospinal fluid of a patient with bipolar psychosis and acute encephalopathy. Pathobiology. 1996; 64:64-6.
13 Martin W J. Stealth viral encephalopathy: report of a fatal case complicated by cerebral vasculitis. Pathobiology. 1996; 64:59-63.
14 Martin W J. Genetic instability and fragmentation of a stealth viral genome. Pathobiology. 1996; 64:9-17.
15 Martin W J. Severe stealth virus encephalopathy following chronic-fatigue-syndrome-like illness: clinical and histopathological features. Pathobiology. 1996; 64:1-8.
16 Martin W J. Stealth virus isolated from an autistic child. J Autism Dev Disord. 1995; 25:223-4.
17 Gollard R P, Mayr A., Rice D A, Martin W J. Herpesvirus-related sequences in salivary gland tumors. J Exp Clin Cancer Res., 1996; 15: 1-4.
18 Martin W J., Glass R T. Acute encephalopathy induced in cats with a stealth virus isolated from a patient with chronic fatigue syndrome. Pathobiology. 1995; 63:115-8.
19 Martin W J, et al. African green monkey origin of the atypical cytopathic ‘stealth virus’ isolated from a patient with chronic fatigue syndrome. Clin Diag Virol 1995: 4: 93-103.
20 Martin W J. Stealth viruses as neuropathogens. CAP Today. 1994; 8: 67-70.
21 Martin W J. et al. Cytomegalovirus-related sequence in an atypical cytopathic virus repeatedly isolated from a patient with chronic fatigue syndrome. Am J. Pathol. 1994; 145: 440-51.

### STATEMENT REGARDING FEDERALLY SPONSORED RESEARCH OR DEVELOPMENT

Not applicable: No Federal funding was received in support of this patent application.

### REFERENCE TO SEQUENCE LISTING, A TABLE OR A COMPUTER PROGRAM LISTING COMPACT DISK APPENDIX

Not applicable.

### BACKGROUND OF THE INVENTION

The invention is based on the following broad conceptual understanding on how the body can acquire cellular energy other than through the oxidative metabolism of foods. Essentially, an alternative cellular energy (ACE) pathway has been identified that is mediated by the energy converting (transducing) properties of mineral containing complexes of organic molecules, arbitrarily termed alternative cellular energy pigments (ACE pigments). Cellular energy generated by this pathway can seemingly complement the chemical energy that is derived by living organisms from the metabolism of food. The ACE pathway is likely to contribute to various physiological functions of the body. Of particular relevance to this application, the ACE pathway is postulated to provide an auxiliary defense mechanism beyond that of the immunological system, such that an inadequacy of this pathway may limit the body's capacity to overcome various infectious diseases. The ACE pathway is also anticipated to be involved in the normal functioning of many organs, including the brain. Moreover, it is reasonable to presume that many illnesses, not necessarily of infectious origin, may place an added burden on the ACE pathway and that an inadequacy or deprivation of the ACE pathway may be a factor in delaying the normal disease recovery process. Conversely, augmenting or activation of an impaired ACE pathway may facilitate recovery from a wide range of various illnesses. Moreover, a fully functioning ACE pathway is likely to also be a factor in maintaining wellness, enhancing athletic performance, increasing cognitive abilities, etc.

ACE pigments are envisioned as tiny batteries that can be either fully charged or not fully charged with energy. In the latter state, ACE pigments can accept additional energy from various sources, including ultraviolet (UV) light. Absorption of UV light energy is observed as the emission of visible light; a process known as fluorescence. In some patients, including patients in whom particles of ACE pigments are misdiagnosed as parasites (delusional parasitosis of Morgellons disease) direct fluorescence of ACE pigments can be readily observed. In many patients, however, the absorption of UV light energy and the resulting fluorescence by inadequately charged ACE pigments can be strongly enhanced in the presence of certain dyes, including neutral red. ACE pigments can also interact with various other dyes, including the fluorescent dye acridine orange, yielding a multiplicity of colors that are well beyond the narrow spectrum of green light emitted solely by UV illuminated acridine orange. Once a fluorescence reaction is evoked, nearby ACE pigments, without any direct contact with the dye, will also commonly fluoresce, presumably through a physical energy transfer mechanism. This process, triggered by neutral red dye, has been successfully used in expediting the healing of active skin lesions caused by herpes simplex virus (HSV), herpes zoster virus (HZV) and human papillomavirus (HPV) infections. It has also been successfully used in preventing future outbreaks in patients with a prior history of recurrent herpes who were treated during a period when no active lesion was present.

Patients with various other illnesses will commonly display ACE pigments in skin and body secretions that will fluoresce under UV light illumination in the presence of freshly prepared solution of neutral red dye. Some of these additional illnesses have been attributed to infections by stealth adapted viruses that fail to provoke an anti-viral cellular immune response. Major categories of these illnesses include the chronic fatigue syndrome, fibromyalgia, psychiatric and both acute and chronic neurological illnesses, autism and both behavioral and learning disorders in children, some cases of cancer and many cases of general debilitating illness. Material that fluoresces upon the addition of neutral red can also be seen in saliva and urine of various patients. These findings have led to the development of simple methods to assess a patient's ACE pathway and to provide a clinical indication for efforts to recharge this pathway. Such efforts include the application of neutral red, either directly to the patient's skin or onto an absorbent cloth on which ACE pigments were collected. The neutral red stained, ACE pigment containing absorbent cloth can be placed on the patient's skin, such that there is no direct contact of the dye with the patient's skin. Using either method, the UV inducible fluorescence will typically extend to involve other areas of the body, consistent with an overall systemic activation of the body's ACE pathway. Beyond developing a simple means to assess the energy status of the ACE pathway in patients, the important question arose as to whether methods of activating the ACE pathway in patients with a variety of illnesses can provide clinical benefit to patients well beyond the therapy of active herpes skin lesions. The answer is affirmative leading to the discovery of a general principle and a specific method to enhance vitality and wellness of patients in whom fluorescing ACE pigments are present.

The present invention includes reports of clinical benefits in several children with autism, one of whom also has an ACE pigment producing illness described as Morgellons disease. The therapy was also effective in treating several women with cellulites.

### BRIEF SUMMARY OF THE INVENTION

The invention describes a method to enhance the overall vitality and wellbeing of individuals though a method of energy-based activation of an alternative cellular energy (ACE) pathway. Alternative cellular energy pigments (ACE-pigments) can be judged as not being fully charged if they fluoresce under UV light illumination when contacted with freshly prepared neutral red dye. The lack of detectable fluorescing material from the skin, buccal swabs (saliva) or urine is presumptive evidence that the ACE pathway is either fully charged or not being called upon to the extent it is in patients in whom fluorescence material can be collected. A series of patients have been investigated in whom skin-derived, neutral red inducible-UV fluorescing material was present but no actual skin disease was observed. Additional material was subsequently collected onto an absorbent cloth, which was stained on one side with neutral red, placed over the skin areas from which the material was collected, and illuminated with UV light. Fluorescence extended to involve the patient's skin. The patients so treated consistently reported and also displayed improvements in their sense of wellbeing. This approach provides a novel method to enhance the health and fitness of individuals in whom the ACE pathway is seemingly not fully charged. It is being referred to as “jump starting” the ACE pathway.

Various natural products possess ACE pigment like activities and have been termed “enerceuticals” by the inventor. They are defined as materials that are capable of transducing (converting) physical energies into an energy form that can be utilized by living cells as an alternative to chemical energy derived from the metabolism of nutrients and which have the following distinguishing characteristics: i) Can provide therapeutic benefits as well as a general vitality enhancing effects to plants, animals and humans; ii) their potential therapeutic effects are not limited or restricted to any particular type or grouping of illnesses; and iii) that do not need to specifically localize to the disease tissues since they can generate field effects that extend beyond their physical location. These criteria distinguish enerceuticals from both pharmaceuticals and neutraceuticals.

An example of an enerceutical is a terpene/terpenoid rich mixture of steam-extracted sap from Japanese cedar, cypress and pine trees and plantain plants. It is marketed as HB-101 for agricultural use and as EH-101 when used as a dietary supplement in humans. This product was simply regarded as a nutrient until the inventor drew attention to its remarkable property of still being active at 1:10,000 dilutions, delaying the spoiling of caught whole fish even when simply included in the ice used to pack the intact fish, and interacting in vigorous energy exchange reactions with tincture of iodine, leading to products that can have distant effects on microbes. Another enerceutical product was formulated from xylocalne (Lidocaine) by the addition of sodium chloride depleted mineral salts obtained from the Great Salt Lake in Utah and marketed by Marine Minerals, Inc. Electrostatically active, needle-shaped crystals form that can both directly and after interaction with tincture of iodine, affect the behavior of microbes placed in a separate water droplet on the same microscope slide as the crystals. Various herbal products have been added to this basic formulation that enhance the formation of electrostatically and energy active Lidocaine-containing complexes. This product has been provisionally designated Epione. It is preferred to HB-101 because it gives a bright UV light induced orange fluorescence when mixed with freshly prepared neutral red. Xylocalne by itself will not fluoresce when mixed with neutral red. Nor will this enerceutical when used without contact with neutral red.

The present invention relates to combining this enerceutical with neutral red to create a UV fluorescing solution that can be applied either directly to the skin or separated from the skin by an impermeable barrier; and illuminating the enerceutical-dye combination with a UV light source, so as to activate the ACE pathway in an ACE pathway deprived human or animal. Once the reaction is triggered, the enerceutical can acquire direct fluorescing properties. Clinical examples are cited of improvements occurring in several autistic children, one of whom had coincidental oral HSV. The use of the UV illuminated enerceutical, either with or without the addition of neutral red for the therapy of cellulites is also described.

### BRIEF DESCRIPTION OF THE DRAWINGS

Not Applicable and none included

### DETAILED DESCRIPTION OF THE INVENTION

Rather than relying upon neutral red to interact directly with ACE pigments derived from a patient, I have discovered that various enerceutical products can be used, either with or without neutral red, to help initiate the activation of the body's own ACE pigments. In a preferred embodiment, a mixed enerceutical-neutral red solution is used to moisten a surgical towel and the towel is placed on one or more skin areas of a patient. If a patient has an existing skin lesion or a prior history of recurrent skin eruptions, the involved areas of skin will first be tried. The towel is then illuminated with a UV light source. Using either the same or another UV light, additional skin areas are subsequently surveyed for fluorescence. In many patients, it is not uncommon to observe additional fluorescence in the axilla, elbows, popliteal fossa, groin, hands, feet, and both the post auricular areas and the hairline of the head. Additional towels moistened with either the enerceutical-neutral red solution, or simply with the enerceutical product by itself, are then placed over the areas of skin fluorescence, and the UV illumination continued for 30-60 minutes, or until the skin fluorescence fades. Incidentally, the palate will also commonly show UV inducible fluorescence during the procedure with subsequent fading.

A suitable enerceutical is the xylocalne-mineral-herbal formulation initially mixed in an equal amount with freshly dissolved neutral red at a concentration of approximately 1 mg/ml. This source of minerals used can be that provided from the Great Salt Lakes and referred to as ionic minerals. It is essentially, the material left in solution after precipitating most of the sodium chloride by simple evaporation. The herbs used include several reportedly present in homeopathic products such as HANSI from Argentina and Canova from Brazil. The actual composition is less important that the ability to i) yield a vigorous reaction when mixed with tincture of iodine and ii) fluoresce under UV light when mixed with neutral red dye at around 0.5 mg/ml.

It was shown that UV illumination of the combination of this enerceutical with neutral red was far more effective in activating skin fluorescence in susceptible individuals than using neutral red alone. Moreover, once skin fluorescence is triggered, the enerceutical by itself, whether on the towel, or placed directly onto the skin, will not uncommonly fluoresce and that this reaction appears to further enhance the body's own UV induced fluorescence response. Indeed, in some patients, the enerceutical used alone with UV illumination, will activate the body's fluoresce, especially as seen on the palate using a UV light, even though in this situation no actual fluorescence of the enerceutical is observed.

Various patients have been treated using the revised protocol comprising moistening one side of an impermeable surgical towel with freshly prepared neutral red solution (at approximately 0.5-1.0 mg/ml); adding an approximately equal amount of the enerceutical to the moistened towel; placing the towel onto affected parts of the body so that a barrier remains between the dye-enerceutical mixture and the patient's skin; followed by UV illumination of the towel and adjacent areas of the patient's skin. Typically a 13 Watt spiral BLB black light is used to illuminate the surgical towel. It can also be used to survey other areas of the patient's skin and inside the patient's mouth, etc. UV illumination of the surgical towel is continued until it is either concluded that i) the body is not going to yield a skin and/or palate fluorescence reaction; ii) skin and/or palate fluorescence reaction having occurred, it is now showing marked fading in its intensity or iii) the fluorescence continues through the available time slot, in which case a subsequent appointment is made for continued illumination. In some patients, three 1 hr sessions were required before the patient became non-fluorescing. For most patients a single 30-60 minute period of UV illumination is adequate.

Using this and related protocols, significant clinical improvements have been seen in patients with a variety of illnesses. The patients have included 3 children with autism, all of whom should significant behavioral improvements following a therapy session. One of these children had concurrent persisting oral herpes simplex virus (HSV) lesions which, as anticipated, scabbed over within a day and fully healed within 3 days. Another autistic child had a long history of skin irritation with the sense of crawling bugs and the formation of skin fibers, referred to as Morgellons disease. This sense of irritation was completely alleviated by the UV light therapy. Most impressive, was the remarkably improved behavior and communicative skills of the youngest of the 3 autistic children. Her mother commented upon how much better behaved she was on returning from the clinic and how over the next several days, the child was more responsive with direct eye contact and had much improved speaking and performance skills. A second session was given to the child who became fluorescence negative when examined on day 3.

An incidental observation in an adult female patient was the marked improvement in cellulite. This led to testing additional patients using a modified tanning bed as the UV light source. UV light illumination of an enerceutical solution applied by a patient directly to her skin, without the addition of neutral red has also been seen. While no fluorescence of the applied enerceutical was observed in the absence of neutral red, her palate was not specifically examined. In other patients, UV illumination of skin applied enerceuticals can provoke a systemic fluorescence, most readily observed by UV illuminating the palate and the back of the throat.

The beneficial effect on cellulite was anticipated from the following reasoning. Cellulite refers to unsightly appearance of skin that has been likened to that of an orange peel or mattress. This appearance results from the formation of numerous, closely spaced shallow dimples causing an uneven, slightly bulging skin surface between the dimples. Cellulite has a nodular, lumpy feel when the underlying tissue is gently pinched.

The basic pathology of cellulite comprises the intrusion of small fatty deposits within the lower dermis (reticular dermis) with the associated formation of fibrous strands that tether the dimpled epidermis to the subcutaneous tissue. The fibrotic reaction is thought to be secondary to the misplaced fat. Cellulite is far more common in women than in men. It has a propensity to form in the upper thighs and buttocks, but can also occur on the abdomen, arms and elsewhere, especially in obese individuals.

I have reasoned that various types of illnesses, including persisting viral infections, can lead to an excess production of fatty acids in the liver, muscle and other cells as a result of mitochondria dysfunction. The body is likely to deposit the excess fat in the subcutaneous tissue, as an unsightly but necessary waste disposal mechanism. I have also reasoned that the deposited fat leads to the proliferation of pre-adipocytes that are formed to help contain the fat that is otherwise irritating. This reasoning came from observing a plastic surgeon collecting abdominal fat for subsequent injection into the cheeks of a gaunt appearing HIV positive patient. The plastic surgeon informed me that it was not uncommon for him to store the collected fat in a−20° C. freezer before using portions to help reshape the face. I confirmed that the freezing process left no viable cells and, therefore, he was essentially inoculating free fat, with the anticipated subsequent formation of new adipocytes from their precursor cells. In some patients the inoculated fat stays as lumpy nodules not unlike that of cellulite.

I, therefore, postulated that cellulite was primarily a failure of adipocyte formation, along with a misplacement of fat causing secondary irritation and fibrosis. It is reasonable, therefore, to expect that individuals deprived of a fully functional ACE pathway might be able to use additional cellular energy to remodel cellulite affected skin.

Using similar reasoning, Institutional Review Board (IRB) approval is being sought to determine potential benefits of ACE pathway activation in other common skin diseases, including male androgenic alopecia (baldness), alopecia areata, psoriasis, age spots, etc. Other patients are being sought with diagnoses of human immunodeficiency virus (HIV), hepatitis C virus (HCV), cytomegalovirus (CMV) infections, etc., as well as patients diagnosed with neurodegenerative illnesses, such as Parkinson's disease, Alzheimer's disease, amyotrophic lateral sclerosis (ALS), multiple sclerosis, etc. Cancer patients are also being sought for clinical studies.

Clearly, clinical protocols based on the knowledge of the ACE pathway have potential value in the therapy of many illnesses, as well as in boosting overall wellness. The protocols are applicable to all individuals in whom there is apparent deprivation of ACE, as evidenced by the appearance of skin, palate and/or urine fluorescence using methods described in co-pending patent applications. The extension of the research is the use of ACE products, termed cenerceuticals, as an adjunct to the dye/UV light protocol described in the co-pending patent applications. The upgraded protocol has been applied to patients with autism, as well as in the repair of cellulite.

The principles, preferred embodiments and modes of operation of the present invention have been described in the foregoing specification. The invention which is intended to be protected herein, however, is not to be construed as limited to the particular enerceutical or dye disclosed, since they are to be regarded as illustrative rather than restrictive. Nor is the invention intended to be necessarily restricted to UV light as the source of physical energy. In some protocols, exposure to sunlight may suffice. Moreover, it is likely that portions of the electromagnetic spectrum other than UV light will prove to be useful with enerceuticals, with or without the inclusion of a dye, for activation of the ACE pathway in an ACE energy-deprived subject. Nor is the invention dependent upon the precise decision as to what does or does not constitute an enerceutical. Basically, the claims relate to the broader issue of achieving therapeutic benefit through activation of the ACE pathway with the description of the currently preferred and practiced method. Additional advantages and modifications will readily occur to those skilled in the art and especially upon practicing the currently described methods. Variations and changes may be made without departing from the spirit of the invention encompassed by the appended claims.

What I claim as my invention is:

1. A method for activating the alternative cellular energy (ACE) pathway in an ACE energy deprived human or animal subject comprising the application of ACE products, termed enerceuticals, to the skin or to a fabric that is placed onto the skin, without or with the addition of a suitable dye solution that can activate ultraviolet (UV) light inducible fluorescence of the enerceutical; such that UV illumination of the enerceutical or enerceutical-dye mixture will evoke UV inducible fluorescence in areas of the skin and/or mucus membranes of the subject with the goal of assisting in the healing of a disease process affecting the subject.

2. A method of accessing the status of the alternative cellular energy (ACE) pathway in a human or animal subject by employing the method of claim 1 and determining whether this method induces the appearance of UV inducible fluorescence within the skin and/or mouth of the subject, such that the appearance of skin or palatal fluorescence is indicative of ACE energy deprivation in the subject.

3. The method of claim 1 in which the enerceutical is a solution containing xylocalne, minerals and herbal extracts formulated in such a manner that it fluoresces under UV illumination when mixed with neutral red at a concentration of approximately 0.5 mg/ml.

4. The method of claim 1 in which the enerceutical is a terpene/terpenoid rich mixture of aqueous steam-extracted solutions from Japanese cedar, cypress and pine trees and plantain plants, and that is marketed as a plant growth product in the United States as HB-01 and marketed as a dietary supplement in the United States as EH-101.

5. The method of claim 1 in which the dye is a solution of neutral red at a concentration ranging from 0.05 to 5.0 mg/ml.

6. The method of claim 1 in which the disease is caused by herpes simplex virus (HSV), herpes zoster virus (HZV), Epstein-Barr virus (EBV), cytomegalovirus (CMV) and human herpes viruses 6-8 (HHV6, HHV7 and HHV8) in a human subject.

7. The method of claim 1 in which the disease is caused by human papillomaviruses

8. The method of claim 1 in which the disease is caused by human immunodeficiency virus (HIV).

9. The method of claim 1 in which the disease is caused by human hepatitis viruses.

10. The method of claim 1 in which the diseases is caused by atypical (stealth adapted) viruses that lack antigenic components capable of activating an effective anti-viral cellular immune response

11. The method of claim 1 in which the diseases is characterized by the formation on the skin and hair of alternative cellular energy (ACE) pigments having the form of particles and fibers and that has been referred to as Morgellons disease

12. The method of claim 1 in which the disease is autism or a related illness, including childhood learning and behavioral disorders.

13. The method of claim 1 in which the disease is the chronic fatigue syndrome or a related illness, including fibromyalgia and depression.

14. The method of claim 1 in which the disease is the skin disorder cellulite
